(19) United States
(12) Patent Application Publication
(10) Pub. No.: US 2007/0292367 A1
(43) Pub. Date: Dec. 20, 2007

(54) METHOD AND SYSTEM FOR FACILITATING AND MAINTAINING ORAL HEALTH THROUGH PRESCRIBED APPLICATIONS OF ORAL COMPOSITIONS

(76) Inventors: Tim Clarot, Phoenix, AZ (US); Regina Miskewitz, Phoenix, AZ (US)

Correspondence Address: SNELL & WILMER L.L.P. (Main), 400 EAST VAN BUREN, ONE ARIZONA CENTER, PHOENIX, AZ, 85004-2202, US

(21) Appl. No.: 11/749,109

(22) Filed: May 15, 2007

Related U.S. Application Data
(60) Provisional application No. 60/800,631, filed on May 15, 2006.

(51) Int. Cl. A61K 8/21; A61K 8/18; A61K 8/22; A61K 8/23; A61K 8/24; A61K 8/27; A61K 8/97; A61Q 11/00

(52) U.S. Cl. 424/52; 424/49; 424/53; 424/56; 424/57; 424/58

(57) ABSTRACT

A system and method for facilitating and maintaining oral health care through regular applications of oral compositions are provided. An exemplary system for facilitating oral health comprises a composition configured to prevent and/or reduce tartar, plaque, gingivitis or gum diseases and/or otherwise facilitate oral health or hygiene and a plurality of containers, each of the containers configured for sealed storage of prescribed dosages of the composition and selective application of the composition to an oral cavity interface, such as the gums, teeth or gingival surface and/or any dental device. The composition can comprise various colors, viscosities, and active ingredients to facilitate improved oral health. A container can also be configured in various manners for application of the composition to an oral cavity interface. A system can also include a storage system configured for storage of the plurality of containers and tracking of usage.

### CROSS REFERENCE TO RELATED APPLICATIONS

This application claims the benefit of U.S. Patent Application Ser. No. 60/800,631, entitled METHOD AND SYSTEM FOR FACILITATING AND MAINTAINING ORAL HEALTH THROUGH PRESCRIBED APPLICATIONS OF ORAL COMPOSITIONS, filed May 15, 2006.

### FIELD OF INVENTION

The present invention generally relates to the maintaining of oral health and hygiene. More particularly, the invention relates to methods and systems for facilitating and maintaining oral health through prescribed applications of oral compositions configured to reduce tartar, plaque, gingivitis, and/or gum disease.

### BACKGROUND OF THE INVENTION

Plaque can be defined as an organized, coherent, gel-like or mucoid material that includes microorganisms in an organic matrix derived from saliva and extracellular bacterial products such as glucans, fructans, enzymes, toxins, and acids. Plaque may also contain other cells, such as desquamated epithelial cells, and inorganic components, such as calcium and phosphate. In general, dental plaque is a bacterial accumulation. Generally transparent and sticky, plaque accumulates around the teeth at the cervical margin, and then grows apically.

Once plaque forms on a surface, the plaque resists removal, and usually can be removed only by mechanical means, such as, for example, by brushing and flossing the affected areas. If not removed, however, the presence of plaque can give rise to tartar formation, tooth decay, gingivitis, periodontitis, and other health problems.

Tartar is a hard, calcified plaque material that exhibits a yellowish or brownish color. Tartar forms as a result of minerals (e.g., those present in saliva and gum pockets) reacting with plaque material to form a rough calculus. Calculus generally arises from the nucleation of calcium phosphate, often in areas where the large salivary gland ducts secrete their saliva. As such, calculus can form on surfaces not covered by the oral mucosa (supragingival) or on surface located apical to the soft tissue margin of the gingiva (subgingival).

Tartar adheres to hard surfaces such as enamel, roots, and dental devices, such as dentures, bridges, crowns, and the like, and is generally more difficult to remove than plaque. Brushing and flossing are normally not sufficient to remove tartar from a surface.

If left untreated, tartar buildup can be problematic in several regards. For example, the rough, porous surface of tartar serves as a breeding ground for additional bacteria, which can calcify and form additional tartar. The bacteria growth can, in turn, lead to tooth decay, gum disease, tooth loss, as well as systemic health problems.

In addition to the health concerns, tartar is a cosmetic problem due to its discoloration of teeth. Namely, teeth can become yellowish or brownish color. Moreover, because the surface of tartar is rough and porous, the tartar absorbs colors from other sources (e.g., coffee, tea, tobacco, smoke, red wine and the like), and thus the presence of tartar exacerbates cosmetic tooth coloration typically associated with such other sources.

Typical methods of preventing tartar buildup include brushing with tartar control toothpaste. Although such toothpastes, if used regularly, may prevent additional buildup of tartar, the toothpastes are not thought to be effective at removing existing tartar from tooth and device surfaces.

Methods of removing existing tartar typically include scaling or root planing, both of which are performed by dentists or hygienists with the aid of specialized tools. Although these techniques work well, they are relatively expensive and time consuming. Furthermore, various methods for inhibiting tartar may cause damage to tooth enamel and/or to dental devices.

### SUMMARY OF THE INVENTION

In accordance with various aspects of the present invention, methods and systems for facilitating and maintaining oral health through regular applications of oral compositions are provided. The various exemplary oral health care systems and methods can provide a relatively inexpensive and safe treatment for facilitating improved oral health and/or hygiene, such as through the reduction of tartar, plaque, gingivitis or gum diseases. In addition, the various exemplary oral health care systems and methods can be relatively easy to use or perform, do not require a visit to a dentist office, and do not damage the surface of enamel.

In accordance with an exemplary embodiment, a system for facilitating and maintaining oral health through regular applications of oral compositions comprises a composition configured to prevent and/or reduce tartar, plaque, gingivitis or gum diseases and/or otherwise facilitate oral health or hygiene, and a plurality of containers configured for sealed storage of prescribed dosages of the composition. The composition can comprise various colors, viscosities, and active ingredients to facilitate improved oral health.

The exemplary containers can be configured in various manners for application of the composition to an oral cavity interface or surface. In accordance with an exemplary embodiment, each container is configured to be readily disposable after a selective application of a prescribed dosage of the composition to an oral cavity surface, such as the gums, teeth or gingival surface and/or any dental device. The amount or number of containers can be determined by a prescribed period of treatment, such as a week, month or year supply of dosages of composition, the period between selective applications, and/or an amount of prescribed dosages for each container, for example, a single, daily dose or multiple dosages within a container.

In accordance with an exemplary embodiment, an exemplary system can also comprise a storage system configured for storage of the plurality of containers and a means for tracking usage of the composition. For example, an exemplary storage system can comprise an application tray configured for storage of the containers and to allow for selective removal of a single container for use. Such an application tray can also be configured for identifying and displaying a prescribed period for systematic application of the containers or composition to facilitate compliance with a prescribed treatment plan. In addition, an exemplary storage system can comprise a recordation device for further tracking usage of the plurality of containers, for example, to determine how many containers have been used within a prescribed period of treatment, and/or how many containers are remaining to be used within the prescribed period. The recordation device can also comprise other related information about the composition and/or treatment plan. In accordance with an exemplary embodiment, the recordation device can be configured within the application tray.

In accordance with an exemplary embodiment, a method of facilitating and maintaining oral health through regular applications of oral compositions includes providing prescribed dosages of composition within a plurality of containers stored in an application tray, retrieving a single container and accessing the composition within, and selectively applying the composition to an oral cavity interface and discarding the used container.

### BRIEF DESCRIPTION OF THE DRAWINGS

The exemplary embodiments of the present invention will be described in connection with the appended drawing figures in which like numerals denote like elements and:

FIG. 1. 1illustrates a block diagram of an exemplary system for facilitating and maintaining oral health care through regular applications of oral compositions in accordance with an exemplary embodiment of the invention;

FIG. 2 illustrates a block diagram of an exemplary container for facilitating and maintaining oral health care through regular applications of oral compositions in accordance with an exemplary embodiment of the invention;

FIGS. 3A, 3B and 3C illustrate, respectively, a side view, a front view and a tilted view of an exemplary container system in accordance with an exemplary embodiment of the invention;

FIGS. 4A and 4B illustrate, respectively, a front view and left view of an exemplary container system in accordance with an exemplary embodiment of the invention;

FIGS. 5A-5D illustrate, respectively, left view, front view, right view and a top view of an exemplary container system in accordance with an exemplary embodiment of the invention;

FIGS. 6A and 6B illustrate a perspective and top view of an exemplary storage system for facilitating and maintaining oral health care through regular applications of oral compositions in accordance with an exemplary embodiment of the invention; and

FIG. 7 illustrates a block diagram of an exemplary method for facilitating and maintaining oral health care through regular applications of oral compositions in accordance with an exemplary embodiment of the invention.

### DETAILED DESCRIPTION

The present invention may be described herein in terms of various functional components, compositions and processing steps. It should be appreciated that such components, compositions and steps may be realized by any number of structural components configured to perform the specified functions. For example, the present invention may employ various plastics, composites, alloys or any combination thereof, to provide the exemplary containers and applicators disclosed herein. In addition, the present invention may be practiced in any number of health care contexts and that the exemplary embodiments relating to a method and system for facilitating and maintaining oral health care through regular applications of oral compositions as described herein are merely indicative of exemplary applications for the invention. For example, the principles, features and methods discussed may be applied to any health care treatment or application wherein a composition for improved health care is desired.

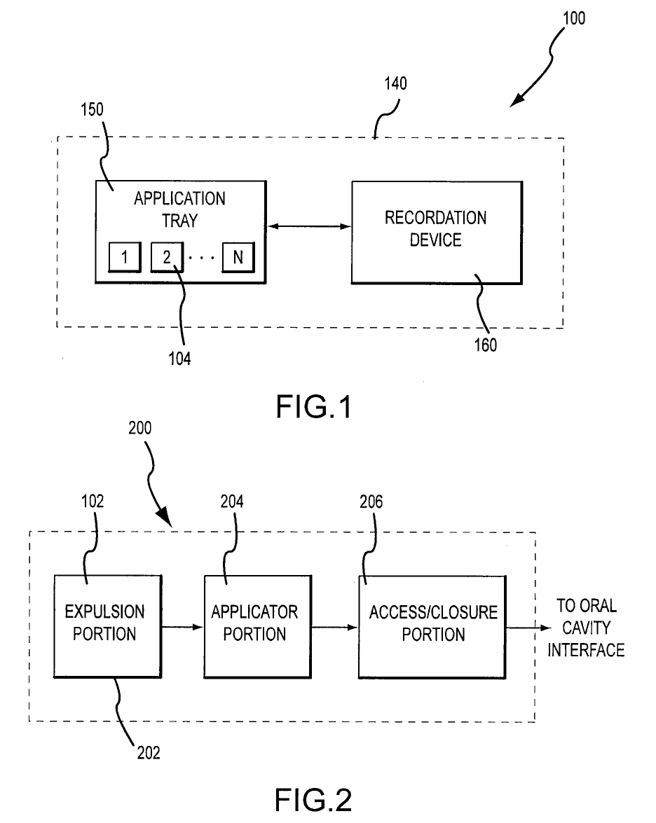

In accordance with various aspects of the present invention, an exemplary oral health care system and method are provided that can facilitate and maintain oral health through regular applications of oral compositions that are configured for the prevention and/or reduction of tartar, plaque, gingivitis, gum diseases and/or otherwise improve oral health and hygiene. In accordance with an exemplary embodiment, with reference to FIG. 1, a system 100 for facilitating and maintaining oral health care comprises a composition encased in a plurality of containers 104 configured for treating an oral cavity interface or surface. The composition comprises ingredients configured to prevent and/or reduce tartar, plaque, gingivitis or gum diseases and/or otherwise improve oral health and hygiene. Container 104 is configured for containment and temporary storage of the composition, i.e., storage until initiation of the treatment process, and for expulsion and application of the composition to oral cavity interface to achieve improved oral health and/or hygiene. The oral cavity interface or surface suitably includes any surface in which oral health care and/or hygiene is desired, such as the gums or gingival surface, the teeth (both supragingival and subgingival) and/or any dental devices such as bridges, crowns, fillings, braces, and the like.

Each one of the plurality of containers 104 is configured to be readily disposable after a selective application of a prescribed dosage of the composition to oral cavity interface, such as the gums, teeth or gingival surface and/or any dental device. The amount or number of containers, i.e., 1, 2, . . . or N, can be determined by a prescribed period of treatment, such as a weekly, monthly or yearly supply of dosages of the composition, the period in between selective applications, e.g., morning and evening/night, daily or two-day or longer periods, and/or an amount of prescribed dosages for each container 104, for example, a single, daily dose or multiple dosages. In accordance with various aspects of a preferred embodiment of the present invention, each container 104 comprises a single-dose delivery mechanism, such that the composition, which is preferably in a useable form without additional treatment or handling, can be readily applied, and thereafter, any used container 104 may be discarded.

Exemplary containers 104 can comprise various sizes and volumes depending on treatment applications, and/or various shapes and configurations for facilitating delivery of the composition. In accordance with an exemplary embodiment, each container 104 is suitably configured to encase or otherwise contain a single dose of the composition, such as a daily dose, to be applied to a selected oral cavity interface. For example, container 104 can comprise a disposable device that comprises a prescribed amount of composition depending on the application, e.g., depending upon whether the treatment process is being applied to the gums or gingival surface, the teeth and/or any dental devices. In accordance with other exemplary embodiments, container 104 can be suitably configured to encase or otherwise contain multiple doses of the composition, such as doses used either within a shorter period of time, e.g., over a day or two, or doses used (and restored) for use over a longer period of time, e.g., over the course of a week or longer.

In accordance with an exemplary embodiment, with additional reference to FIG. 1, an exemplary system 100 can also comprise a storage system 140 configured for storage of the plurality of containers 104 and for tracking of prescribed usage. For example, an exemplary storage system 140 can comprise an application tray 150 configured for storage of containers 104 and to allow for selective removal of a single container 104 for prescribed use. In accordance with an exemplary embodiment, application tray 150 can also be configured for identifying and displaying a prescribed period for systematic application of containers 104 to facilitate compliance with a prescribed treatment plan. An exemplary storage system 140 can also comprise a recordation device 160 for tracking usage of the plurality of containers 104, for example, to determine how many containers 104 have been used within a prescribed period of treatment, and/or how many containers 104 are remaining to be used within the prescribed period. Recordation device 160 can also comprise other related information about the composition and/or treatment plan. Application tray 150 and recordation device 160 can be configured in various manners for storage of the plurality of containers 104 and for tracking of prescribed usage.

An exemplary container 104 can be configured in various manners for application of the composition to oral cavity interface. For example, with additional reference to FIG. 2, in accordance with an exemplary embodiment, each container 200 can comprise an expulsion portion 202 configured to contain or store composition 102 and to facilitate expulsion of composition 102, and an applicator portion 204 configured to receive composition 102 from expulsion portion 202 and to facilitate application or delivery of expelled composition 102 to a selected oral cavity interface. In accordance with an exemplary embodiment, container 200 substantially comprises a low density, polyethylene material. However, container 200 can comprise various other materials, composites, alloys and the like for providing the structures configured for storage and delivery or application of composition.

Expulsion portion 202 suitably comprises a resilient structure that can contain or store composition 102 for a desired period of time, e.g., hours, days, weeks or longer, and then allow a user to apply a suitable force to deliver or transfer composition 102 into applicator portion 204. In accordance with an exemplary embodiment, a prescribed dosage of composition may reside or otherwise be contained within expulsion portion 202 prior to usage, and then substantially emptied from expulsion portion during use. For example, expulsion portion 202 may store a single dosage of composition for treatment, e.g., a daily dose, or sufficient composition for two or more applications used over a variable prescribed period. Expulsion portion 202 can be suitably filled with composition 102 in various manners. In an exemplary embodiment, expulsion portion 204 is substantially filled with composition 102, and then crimped, heated, folded, capped or otherwise sealed to contain composition in storage until the user is ready to initiate use.

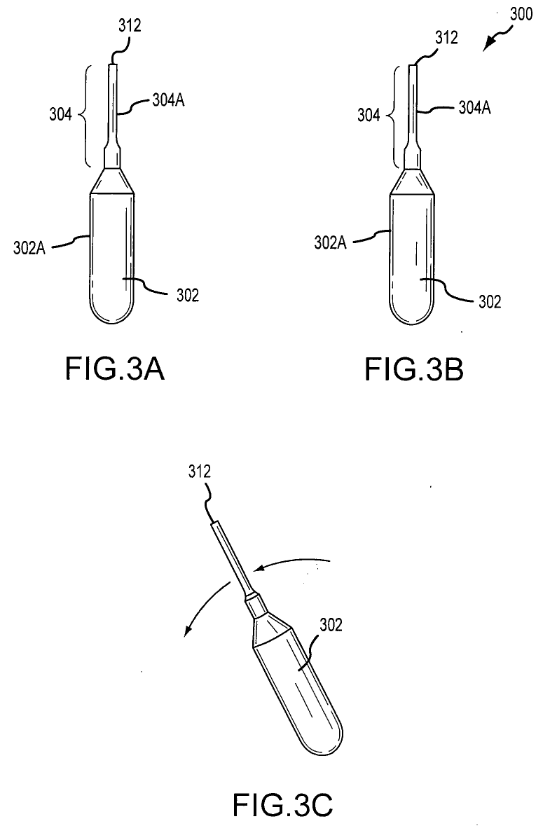

Expulsion portion 202 can also be configured to allow a user to suitably control the rate of expulsion into applicator portion 204. For example, with momentary reference to FIG. 3, in accordance with an exemplary embodiment, a container 300 can include an expulsion portion 302 that can comprise a plastic-type, cylinder or tube-like structure 302A that suitably encloses composition 102 and can be squeezed or otherwise can have a force applied for expulsing composition into an applicator portion 304. For example, a tube-like structure can be easily gripped with the fingers or hand and squeezed to force any composition within to be transferred into applicator portion 304.

While expulsion portion 302 can suitably comprise a cylinder or tube-like shape 302A, expulsion portion 302 can also comprise a rectangular shape, a substantially flat-like shape, e.g., that similar to a hot water bottle, or any other shape that can contain or store compositions and allow for the expulsion of compositions by a force. In addition, while such a force can be suitably applied on an exterior of expulsion portion 302, e.g., similar to “squeezing” a tube of toothpaste approximate a center-section or on one end and rolling or collapsing the tube, such an expulsion force can also be realized by urging a plunger-like device against, e.g., much like a syringe, or any other like manner wherein the amount of force and/or the rate or expulsion or delivery into applicator portion 304 can be suitably controlled.

Applicator portion 204 is suitably configured for selective or otherwise controlled delivery of composition 102 to a target area within an oral cavity interface 106. Applicator portion 204 can suitably comprise a detachable component from expulsion portion 202, or can comprise a molded or otherwise unitary structure with expulsion portion 202. With momentary reference again to FIG. 3, in accordance with an exemplary embodiment, container 300 can include an applicator portion 304 that is suitably molded in a unitary structure with expulsion portion 302. For example, container 300 can comprise an injection molded tube-like structure for portions 302 and 304. In accordance with other embodiments, applicator portion 204 can suitably comprise a separate component that is suitably screwed, press-fit, clamped or otherwise permanently, semi-permanently or removably attached to expulsion portion 202.

In accordance with an exemplary embodiment, applicator portion 204 comprises a passive structure to allow composition 102 to be forced through an applicator tip 312 and onto an oral cavity interface. For example, with reference again to FIG. 3, applicator portion 304 can comprise a semi-rigid or substantially rigid tube-like component 304A configured to allow composition to flow to applicator tip 312 through upon an expulsion force being applied to expulsion component 302. In other embodiments, applicator portion 304 can also be configured as a less-rigid or otherwise expulsive component to allow for any remaining composition within applicator portion 304 to be suitably squeezed or otherwise delivered or applied by applicator tip 312 onto oral cavity interface 106.

In the exemplary embodiment of FIG. 3, applicator tip 312 suitably comprises an opening at a discharge end of applicator 304 to allow for any composition to be discharged or otherwise delivered from the opening and applied to an oral cavity interface. For example, the composition can be discharged from applicator tip 312 and applied to a gum tissue region, such as by tilting container 300 as illustrated in FIG. 3C. As such, composition can be targeted for delivery onto the tissue region at selected amounts and locations to achieve a desired treatment, while avoiding other areas of the oral cavity that composition is not to be applied or needed. In accordance with an exemplary embodiment, applicator tip 312 can suitably comprise a similar shape or configuration to that of portion 304A, e.g., a tube structure; however, applicator tip 312 can also comprise a narrower or wider cross-sectional configuration. For example, tip 312 may include a disposable break-away portion, as will be described in greater detail hereinbelow.

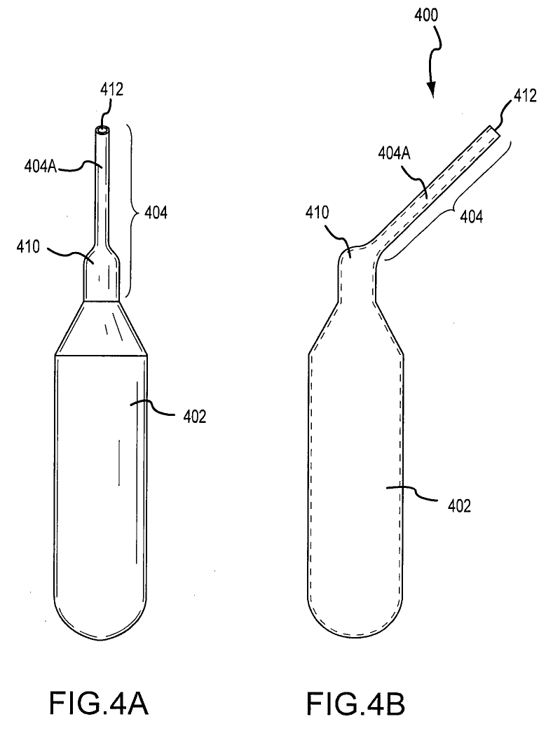

In accordance with an exemplary embodiment, applicator portion 204 can be further configured to facilitate treatment in difficult to reach target areas within the oral cavity interface. For example, with reference to FIGS. 4A and 4B, rather than using substantial tilting to direct applicator portion 304 to an selected oral cavity interface, an applicator portion 404 can suitably comprise an angled portion 410 that allows applicator component 404A to suitably bend towards or otherwise more readily allow a user to reach selected areas of the gums, gingival surface, teeth and/or any dental devices. In accordance with exemplary embodiments, angled portion 410 can be suitably angled upwards, e.g., component 404A can comprise an angle between approximately 90 degrees and 180 degrees with an expulsion portion 402, angled downwards, e.g., component 404A can comprise an angle between approximately 1 degree and 90 degrees with an expulsion portion 402, or an angle at substantially 90 degrees. For example, having an angle upwards or at approximately 90 degrees can more readily facilitate application of composition to an outer interface of teeth, gums or dental devices, while having an angled downwards can more readily facilitate application of composition to an interior surface, e.g., the tissue and gum regions behind teeth or dental devices. Angled portion 410 can also comprise various sizes, shapes and cross-sectional configurations to allow composition to be expulsed from expulsion portion 402 into applicator portion 404 and out of applicator tip 412.

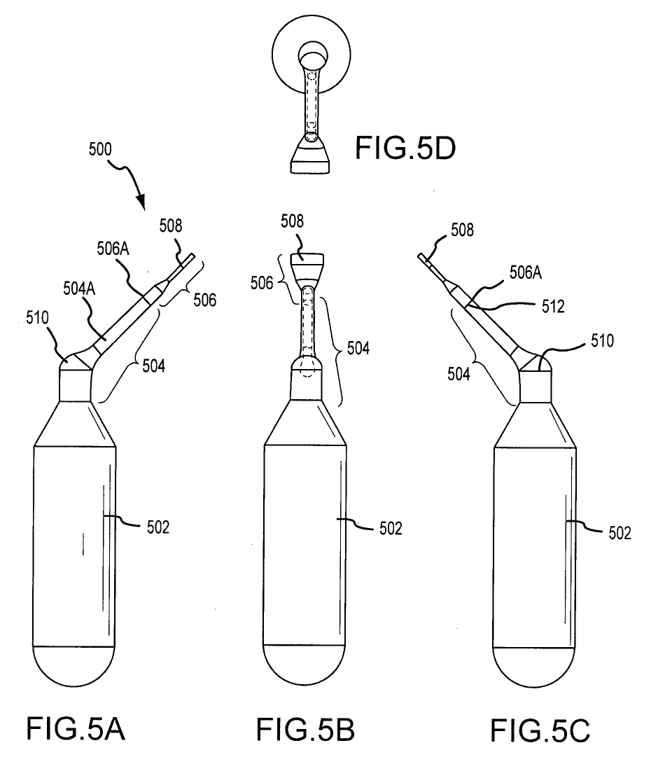

In accordance with an exemplary embodiment, with reference again to FIG. 2, container 200 can also be configured with an access/closure portion 206 to help maintain composition 102 within container 200 and allow release of composition 102 from applicator portion 204. Access/closure portion 206 can suitably comprise a removable component that is suitably disposed when access is desired. For example, with reference to FIGS. 5A-5D, an access/closure portion 506 can comprise a detachable device that suitably separates at a region 506A such that an applicator tip 512 is exposed. To gain access, a user can simply grip and detach, e.g., by pulling, bending, twisting and/or other like separating action, to remove access portion 506 from applicator portion 504. Access portion 506 can be completely detached and removed or discarded, or substantially detached such as by bending backwards to provide an opening and yet remain connected with at least some portion adjacent applicator tip 512.

In accordance with an exemplary embodiment, access portion 506 can suitably comprise a tab component 508 configured to allow a user to simply grip and detach. Tab component 508 can comprise various shapes, thicknesses and sizes for facilitating easier removal of access portion 506 from applicator portion 504. Tab component 508 further comprises a closed end, i.e., without an opening or otherwise configured to facilitate a sealed closure of applicator portion 504. Having tab component 504 can make gripping of access portion 506 easier, although other exemplary embodiments can be configured without tab component 508, such as access portion 506 having substantially the same shape, cross-sectional area and/or configuration of applicator portion 504.

In the exemplary embodiment illustrated in FIGS. 5A-5D, access portion 506 comprises a detachable component that remains detached, e.g., region 506A and applicator tip 512 are not made for re-attachment. Such embodiments may be desirable for single or other short term uses. In accordance with other exemplary embodiments, access portion 206 can comprise a re-attachable configuration in which a detached region can be suitably screwed, clamped, press-fit or otherwise re-attached to applicator portion 204, such as to enable re-use over a period of days. In addition, to prevent misplacement after detaching or opening, access portion 206 can comprise a strap portion that surrounds or is otherwise attached to applicator portion 204 or any other part of container 200 that allows for access portion 204 to be suitably found and used to reseal or close applicator portion 204 after use.

With reference again to FIG. 2, composition 102 can comprise various colors, viscosities, active ingredients and viscous carriers to facilitate prevention and/or reduction of tartar, plaque, gingivitis, gum diseases and/or otherwise facilitate oral health and hygiene within an oral cavity.

In accordance with an exemplary embodiment, composition 102 comprises an active ingredient and a viscous carrier. Composition 102 is configured to maintain the active ingredient in contact with an oral cavity interface for an extended period of time to allow the active ingredient(s) to be activated. An exemplary active ingredient can include one or more of the following: cetylpyridinium chloride (CPC), dicalcium phosphate dehydrate, hydrogen peroxide, sanguinaria extract, sodium bicarbonate, sodium lauryl sulfate, stannous fluoride, zinc salts such as zinc chloride, zinc acetate, zinc citrate, and zinc gluconate, zinc oxide, alkyl dimethyl amine oxide, alkyl dimethyl glycine, eucalyptol, menthol, methyl salicylate, thymol, sodium citrate, peppermint oil, sage oil, polymethylsiloxane, polxamer, and stannous pyrophosphate. Other now known or hereafter devised actives may also be used. For example, any agent which is alone or in combination able to prevent or alleviate the severity of problems associated with dentition may be utilized. Such may include anti-caries agents, such as sodium fluoride, stannous fluoride, sodium monofluorphosphate, and/or the like; agents useful in reducing tooth hypersensitivity, such as potassium nitrate, strontium chloride and/or the like; and/or plaque and calculus reducing agents, such as, for example, chlorhexidine, quaternary ammonium compounds (e.g. benzethonium chloride, domphen bromide, etc.),. triclosan, herbal compounds (e.g. sanguinarine), stannous salts, complex phosphates (e.g., pyrophosphates), SLS (e.g. sodium lauryl sulfate), hydrogen peroxide, and/or the like.

An amount of the active ingredient suitable for use within composition 102 varies in accordance with the dosage size and particular ingredient(s). In general, the active or actives selected will be used in a suitably effective amount, generally on the order of less than about 10 wt %, and more preferably 5 wt % or less. In particularly preferred compositions, the active ingredient may be present in an amount of about 0.001 wt % to about 1.5 wt %, within an amount of about 0.025 wt % to about 1.0 wt %, or even within an amount about 0.05 wt % to about 0.7 wt %. All percentages set forth herein are in weight percent of the total composition, unless otherwise indicated.

In accordance with one preferred exemplary embodiment, the active ingredient comprises CPC. For example, CPC may be present in an amount of about 0.001% to about 1%, in an amount of about 0.01% to about 0.5%, or even in an amount of about 0.05% to about 0.25% or about 0.045% to about 0.1%. In accordance with another exemplary embodiment, the active ingredient can also comprise zinc gluconate. For example, zinc gluconate can be present in an amount of about 0.001% to about 1.5%, in an amount of about 0.01% to about 1.0%, or even in an amount of about 0.05% to about 0.75%.

In accordance with an exemplary embodiment, composition 102 can also include a thickener to obtain the desired viscosity. Suitable thickening agents include substances which increases the viscosity of composition 102, cause composition 102 to gel or coagulate, or the like, such as food-grade or pharmaceutical grade thickeners, including, for example, hydroxyethylcellulose, hydroxypropyl methylcellulose, carrageenan, guar gum, methylcellulose, methyethylecellulose, acceptable non-ionic thickeners, and the like. The thickener may be present in an amount of about 0.01 to about 10%, in an amount of about 0.1 to about 7%, or even in an amount of about 1% to about 5%.

Composition 102 may also include a humectant such a glycerin, which may be present in an amount of about 0.01% to about 15%, preferably about 0.1% to about 10%, and more preferably about 1 to about 7%. When used, the humectant may facilitate maintaining composition 102 in a liquid form and may help maintain a desired viscosity. In addition, glycerin may facilitate maintaining one or more of the active ingredients in an ionic form and may facilitate the transport of the active ingredients through composition 102.

Composition 102 may also include a suitable diluent. Exemplary diluents suitable for use within composition 102 include water, alcohols, and oils. For example, composition 102 can includes purified water in an amount of about 80% to about 99%, in an amount of about 85% to about 95%, or even in an amount of about 88% to about 92%.

Composition 102 may also include sugar alcohols such as sorbitol and xylital, monnital that act as a sweetener and also as a humectant and/or emulsifier. When used, sorbitol or other sugar alcohol can be present in an amount of about 0.001% to about 0.5%, in an amount of about 0.01% to about 0.1%, or even in an amount of about 0.025% to about 0.075%.

Composition 102 may also include a natural or artificial sweetener such as sucralose, saccharin, ace-k, aspartame which, when included in composition 102, can be present in an amount of about 0.001% to about 1.5%, in an amount of about 0.01% to about 1%, or even in an amount of about 0.25% to about 0.75%.

Colorants may also be added to composition 102. For example, composition 102 can includes colorants, such that when composition 102 is applied to or proximate the gingiva, composition 102 has a color indicative of healthy gingiva—e.g., composition 102 can be pink in color. Such a composition having a color indicative of healthy gingiva can provide added incentive to users to continue using composition 102, which in turn promotes improved health care and hygiene. Colorants may be present in any desired amount. For example, the colorants may include Red #33 and Red #40, available from Pylam in an amount of about 0.000005% to about 0.00018%, and more preferably about 0.00050% to about 0.00099% , respectively.

Composition may also include flavorants such as cinnamon oil, clove oil, mints, anise, citrus, fruits, and the like, which, when included in the formula are present in an amount of about 0.001% to about 2%, in an amount of about 0.01% to about 1%, or even in an amount about of about 0.1% to about 0.5%.

The viscosity of composition 102 is configured to maintain composition 102 in contact with an oral cavity interface 106 for an extended period of time, which has several advantages over traditional compositions. Composition 102 preferably exhibits good microadhesion, and moreover, composition 102 preferably is quite viscous. As such, in general, relatively small amounts of composition 102 and consequently the active agent(s) can be used to effectively provide oral health care or treatment. Additionally, the relatively high viscosity allows for relatively select placement of the composition on a surface. Exemplary compositions can comprise a viscosity greater than about 20,000 cp, greater than 30,000 cp, and at least greater than about 35,000 cp. The viscosity values as set forth herein are measured using a Brookfield, Model DV-II+ Pro viscometer, spindle # 6, 10 RPM for 90 seconds at 25 C.

In accordance with another embodiment, composition 102 can comprise multiple active ingredients in a carrier, within and without a thickening agent. For example, composition 102 can include a plurality of any active ingredients and any thickening agents in the weight percents disclosed. Composition 102 may also include any of the optional ingredients such as sweeteners, flavorants, and colorants as set forth herein. For example, in accordance with an exemplary embodiment, composition 102 can comprise CPC and zinc gluconate as the active ingredients, wherein the CPC is present in an amount of about 0.001% to about 1%, in an amount of about 0.01% to about 0.5%, or even in an amount of about 0.05% to about 0.25% or about 0.045% to about 0.1%; and wherein the zinc gluconate is present in an amount of about 0.001% to about 1.5%, in an amount of about 0.01% to about 1.0%, or even in an amount of about 0.05% to about 0.75%.

In accordance with other exemplary embodiments, composition 102 can comprise one or more active ingredients and a colorant indicative of healthy gingival, wherein the color of composition 102 is more than merely decorative to serve the function of encouraging those that use the product to continue to use the product because there is an immediate appearance, upon application of composition 102, that healthy gingival is achieved.

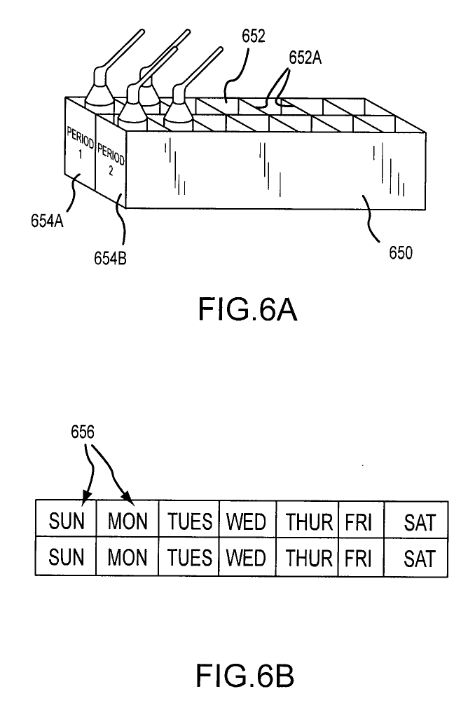

As set forth herein, with reference again to FIG. 1, an exemplary application tray 150 and recordation device 160 can be configured in various sizes, shapes and orientations for storage of the plurality of containers 104 and for tracking of prescribed usage over a variety of prescribed treatment periods. In accordance with various exemplary embodiments, recordation device 160 can be configured within application tray 150, or otherwise integrated into application tray 150. For example, with additional reference to FIGS. 6A and 6B, in accordance with an exemplary embodiment, an exemplary application tray 650 can comprise a two-week daily supply of single-dose containers 604, each such container 604 suitably stored or maintained within a plurality of compartments 652. The size and shape of compartments 652 can be configured in various arrangements, e.g., square or rectangular, circular or other like shapes, to suitably contain containers 604 within during transporting or other uses of application tray 650. In addition, application tray 650, including compartments 652, can comprise various materials, such as paperboard, plastics, thin-alloys, fiberglass, or other composites, or any combination of materials thereof, for providing the storage functions.

To further secure containers 604 within application tray 650, in accordance with an exemplary embodiment, each compartment 652 can be configured with an opening, e.g., the spacing between compartment sides 652A, only slightly larger than an outer circumference or perimeter of containers 604 to facilitate a snug or tight fit when containers 604 are placed within compartments 652. In accordance with another exemplary embodiment, each compartment 652 can also comprise a carved-out, bottom portion having a shape substantially conforming to a bottom shape of containers 604 to facilitate a snug or otherwise secure fit within compartments 652. Such carved-out bottom portions can comprise various paperboards, plastics, e.g., Styrofoam, or other like composites capable of providing a substantially conforming shape with containers 604.

To facilitate tracking of usage of containers 604, an exemplary application tray 650 can be configured for identifying and displaying a prescribed period for systematic application of containers to facilitate compliance with a prescribed treatment plan. For example, in accordance with the embodiment illustrated in FIG. 6A, application tray 650 can comprise one or more period indicators 654A and 654B, e.g., a first week and a second week of a two-week supply, as well as single-dose container indicators 656, e.g., the days of the week. Thus, for example, for a two-week supply of single-dose containers 604 prescribed for daily use, a user can simply retrieve a single container 604 beginning on the first day of the week, i.e., Sunday, and selectively apply to a targeted area of an oral cavity interface, and discard that container 604 after use, followed by retrieval, application and use of remaining containers 604 as the weeks progress until the two-week supply is exhausted.

While the exemplary embodiment of application tray 650 illustrated in FIGS. 6A and 6B comprises a two-week supply, shorter or longer periods of treatment and/or shorter or longer time periods between applications of containers 604 can also be provided within exemplary application trays 650. For example, application tray 650 can be configured to store and maintain a one-week supply, a month supply, three-month supply, or a year-supply, or any other shorter or longer prescribed periods depending on the prescribed treatment plan. In addition, the time period between prescribed uses of containers 604 can be configured for a morning and an evening/nighttime use, e.g., a.m. and p.m. indicators 656, or over periods of every two or three days or longer. In accordance with an exemplary embodiment, application trays 650 can be configured for stacking, e.g., with a top cover surrounding containers 604 and/or with sides 652 extending to a height at least as long as the vertical length of containers 604. Such application trays 650 can be configured for discarding after use of all containers 604 within, or for restocking with new containers 604 ready for selective application of composition.

In the exemplary embodiment, application tray 650 provides an integrated recordation device for further tracking usage of a plurality of containers 604, for example, to determine how many containers 604 have been used within a prescribed period of treatment, e.g., within the exemplary two-week periods 654A and 654B, and/or how many containers 604 are remaining to be used within the prescribed period, e.g., indicators 656 can indicate that three containers 656 have been used, and that four days of containers 604 are remaining. An exemplary recordation device can also comprise a separate component, for example, an information and/or recordation display card that can be suitably folded or otherwise inserted within or coupled to application tray 650 that allows a user to mark-off, e.g., with a writing instrument or through puncture of use indicators configured within the display card.

Regardless of whether integrated within application tray or comprising a separate component, an exemplary recordation device can also comprise other related information about composition 102 and/or a prescribed treatment plan. For example, information regarding the expiration or expectancy of effective use of composition 102 can be suitably configured on application tray 650 or otherwise on a separate display card, as well as a summary or detailed explanation of the prescribed treatment plan and/or instructions on how to selectively apply composition 102 depending on the target area of oral cavity interface 106.

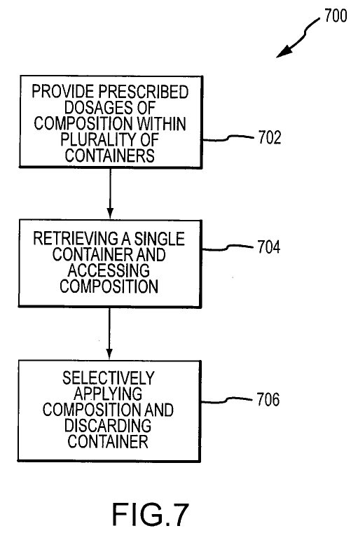

In accordance with an exemplary embodiment, with reference to FIG. 7, as well as continuing reference to FIGS. 1, 3A, 4B, 5A, 6A, and 6B, an exemplary method 700 of facilitating and maintaining oral health care through regular applications of oral compositions can comprise providing prescribed dosages of composition 102 within a plurality of containers 104 (702), retrieving a single container 104 and accessing composition 102 within (704), and selectively applying composition 102 to an oral cavity interface 106 and discarding of used container 104 (706).

Providing prescribed dosages of composition 102 within a plurality of containers 104 can comprise any method now known or hereinafter devised for filling containers with a substantially liquid or other like composition. For example, composition 102 can be suitably filled in one end of containers 104 and then sealed to maintain composition 102 within. In addition, providing composition 102 within containers 104 can preferably comprise providing a single and/or daily dose of composition 102, or even multiple doses of composition 102.

Retrieving a single container 104 and accessing composition 102 within suitably comprises retrieval from a storage system 140 and removal of an access or closure device or component from container 104. For example, in accordance with an exemplary embodiment, retrieval from a storage system 140 can comprise retrieval from an application tray 650, while accessing composition 102 within container 104 comprises detachably removing of an access component, e.g., access portion 506, to provide an applicator tip, such as applicator tip 312.

Selectively applying composition 102 to an oral cavity interface 106 and discarding of used container 104 can suitably comprise expulsing or otherwise forcing or delivering composition 102 from a storage portion to an applicator portion of container 104. For example, in accordance with an exemplary embodiment, composition 102 can be “squeezed” from an expulsion portion, such as expulsion portion 302 or 402, into an applicator portion, such as 304 or 404, through an applicator tip, such as 312 or 412 and onto a targeted region of oral cavity interface 106, such as target region 602A. In accordance with another exemplary embodiment, applying with an applicator portion 304 or 404 can be realized through an angled portion configured to facilitate targeted placement of composition 102. After such use, an exemplary container 104 can be suitably discarded and a next prescribed use can be initiated with a new, filled container 104. As a result, regular and/or systematic use of system 100 at one or more target regions can result in improved oral health, such as the area of tissue regeneration above a tooth.

The present invention has been described above with reference to various exemplary embodiments. However, those skilled in the art will recognize that changes and modifications may be made to the exemplary embodiments without departing from the scope of the present invention. For example, the various operational steps, as well as the components for carrying out the operational steps, may be implemented in alternate ways depending upon the particular application or in consideration of any number of cost functions associated with the operation of the system, e.g., various steps may be deleted, modified, or combined with other steps. These and other changes or modifications are intended to be included within the scope of the present invention, as set forth in the following claims.

1. An oral health care system for facilitating and maintaining oral health through regular applications of oral compositions, said system comprising:
a composition configured to improve oral health, the composition having a viscosity greater than about 20,000 centipoise; and
a plurality of containers configured for sealed storage of prescribed dosages of said composition, each one of said plurality of containers being disposable after selective application of a prescribed dosage of said composition to an oral cavity interface.

2. The oral health care system according to claim 1, wherein an amount of said plurality of containers is determined by a prescribed period of treatment and an amount of said composition within said prescribed dosages.

3. The oral health care system according to claim 2, wherein each of said prescribed dosages comprise a single dose of said composition.

4. The oral health care system according to claim 2, wherein said prescribed period comprises at least a one-week period.

5. The oral health care system according to claim 2, wherein said prescribed period comprises at least a one-month period.

6. The oral health care system according to claim 1, wherein said system further comprises a recordation device for tracking usage of said plurality of containers.

7. The oral health care system according to claim 1, wherein said system further comprises an application tray configured for storage of said plurality of containers and to allow for selective removal of a single container for use and disposal.

8. The oral health care system according to claim 7, wherein said application tray is configured for identifying and displaying a prescribed period for systematic application of said plurality of containers to facilitate compliance with a prescribed treatment plan.

9. The oral health care system according to claim 8, wherein said application tray further comprises a recordation device for tracking usage of said plurality of containers.

10. The oral health care system according to claim 1, wherein said composition is configured to reduce at least one of tartar, plaque, gingivitis and gum diseases within the oral cavity.

11. The oral health care system according to claim 10, wherein said composition comprises an active ingredient and a viscous carrier.

12. The oral health care system according to claim 11, wherein said composition comprises zinc gluconate and cetylpyridinium chloride.

13. The system of claim 11, wherein said composition comprises cetylpyridum chloride, zinc in an ionic state and glycerin.

14. The system of claim 10, wherein said composition has a viscosity of greater than about 30,000 cp.

15. The system of claim 10, wherein said composition has a viscosity of greater than about 35,000 cp.

16. A disposable, self-contained oral health care system configured to reduce at least one of tartar, plaque, gingivitis and gum diseases within an oral cavity, said system comprising:
a composition configured to reduce at least one of tartar, plaque, gingivitis and gum diseases within the oral cavity;
a plurality of containers each being configured for sealed storage of predetermined number of doses of said composition, and being disposable after selective application of a the predetermined number of doses of said composition to an oral cavity interface; and
a storage system configured for storage of said plurality of containers and tracking of usage of said composition within a prescribed treatment plan.

18. The disposable, self-contained oral health care system of claim 17, wherein the composition has a viscosity greater than about 20,000 centipoise.

19. The disposable, self-contained oral health care system of claim 17, wherein the composition comprises an active ingredient selected from the group consisting of cetylpyridinium chloride (CPC), dicalcium phosphate dehydrate, hydrogen peroxide, sanguinaria extract, sodium bicarbonate, sodium lauryl sulfate, stannous fluoride, zinc salts, alkyl dimethyl amine oxide, alkyl dimethyl glycine, eucalyptol, menthol, methyl salicylate, thymol, sodium citrate, peppermint oil, sage oil, polymethylsiloxane, polxamer, and stannous pyrophosphate.

20. A method of facilitating and maintaining oral health care through regular applications of oral compositions, said method comprising:
providing prescribed dosages of a composition within a plurality of containers, said composition configured to reduce at least one of tartar, plaque, gingivitis and gum diseases within an oral cavity;
retrieving a single container from said plurality of containers and accessing said composition within; and
selectively applying said prescribed dosage of composition from said single container to an oral cavity interface and discarding of said single container after said selective application.
